(19) United States
(12) Patent Application Publication
(10) Pub. No.: US 2012/0213827 A1
(43) Pub. Date: Aug. 23, 2012

(54) PROCESS FOR CONTROLLED CRYSTALLIZATION OF AN ACTIVE PHARMACEUTICAL INGREDIENT FROM SUPERCOOLED LIQUID STATE BY HOT MELT EXTRUSION

(76) Inventors: Ashish Chatterji, East Brunswick, NJ (US); Dipen Desai, Whippany, NJ (US); Dave Alan Miller, Round Rock, TX (US); Harpreet K. Sandhu, West Orange, NJ (US); Navnit H. Shah, Clifton, NJ (US)

(21) Appl. No.: 13/398,902

(22) Filed: Feb. 17, 2012

Related U.S. Application Data
(60) Provisional application No. 61/443,743, filed on Feb. 17, 2011.

(51) Int. Cl. A61K 31/265; B29C 47/00; A61K 31/405; A61K 31/167; A61K 9/14; A61K 31/192

(52) U.S. Cl. 424/400; 514/570; 514/420; 514/630; 514/513; 264/176.1

(57) ABSTRACT

A process for controlling the crystallization of certain hydrophobic active pharmaceutical ingredients (APIs) from a supercooled liquid state by hot-melt extrusion processing is described. Also described is a pharmaceutical composition comprising a solid crystalline dispersion of a cholesterol ester transfer protein inhibitor in a hydrophilic excipient matrix. In the process of the present invention, the API is fed to an extrusion system in a crystalline state contemporaneously with carrier excipients where it is first converted to a non-crystalline state by the application of heat and then subsequently recrystallized in-situ by the removal of heat and application of shear. Recrystallization of the API is controlled by carrier formulation design and the hot-melt extrusion process parameters; i.e. barrel temperature profile, feed rate, etc.

### PRIORITY TO RELATED APPLICATIONS

This application claims the benefit of U.S. Provisional Patent Application No. 61/443,743, filed on Feb. 17, 2011, which is hereby incorporated by reference in its entirety.

### FIELD OF THE INVENTION

The present invention concerns a hot-melt extrusion process for reducing the mean particle diameter of certain hydrophobic active pharmaceutical ingredients (APIs) while contemporaneously dispersing said particles in an excipient carrier. The present invention also concerns a pharmaceutical composition comprising a crystalline solid dispersion of a cholesterol ester transfer protein (CETP) inhibitor in an excipient carrier and a method of preparing the same. The hot-melt extruded composition provides rapid dissolution of the API in a use environment (i.e., in the gastrointestinal tract or in an in vitro environment of a test solution, such as simulated gastric fluid, phosphate buffered saline, or a derivative of simulated intestinal fluid).

### BACKGROUND OF THE INVENTION

With the implementation of high-throughput screening in the pharmaceutical industry, the proportion of poorly water-soluble drugs entering into development portfolios has significantly increased. Poor water-solubility limits dissolution of therapeutic compounds in use environments relevant to drug delivery; e.g. in the human gastrointestinal (GI) lumen. With respect to oral delivery of therapeutic compounds, poor water solubility can lead to slow dissolution in the GI tract causing limited absorption and reduced efficacy. Often, high dose administration is the employed strategy to compensate for a low fraction absorbed. However, high inter/intra subject variability and sensitivity to the GI environment (fed/fasted state or diseased state) can also affect oral administration of poorly water-soluble drugs. Therefore, administration of high doses can result in greater incidents of drug-related toxicity associated with excursions above the therapeutic window related to high absorbers or fluctuations in the GI environment.

Consequently, pharmaceutical technologies have been and are continuing to be developed to improve the dissolution properties of poorly water-soluble drugs, including but not limited to the following: salt formation, prodrugs, particle size reduction by attrition methods, solubilized formulations, lipid-based formulations, emulsion systems, molecular complexation, co-crystallization, and solid dispersions. Each of these technologies aim to improve oral delivery of poorly-water soluble drugs by increasing dissolution rates and/or enhancing solubility. The present invention relates to the former in that dissolution of the poorly water-soluble API is increased by in situ API particle size reduction (increased surface area) with simultaneous distribution in a hydrophilic carrier (enhanced surface wetting).

Particle size reduction has been repeatedly demonstrated in the pharmaceutical literature to significantly improve the dissolution rates of poorly water-soluble APIs, correspondingly yielding improved absorption and potentially improved drug therapies. Approaches to particle size reduction can be categorized as either top-down or bottom-up methods. Micronization, wet milling (see, e.g., U.S. Pat. No. 5,494,683) and nano-milling (see, e.g., PCT Int. Appl. WO 2004/022100 and U.S. Pat. Nos. 6,811,767; 7,037,528; and 7,078,057) are examples of techniques that can be applied to poorly water-soluble drugs to reduce particle size by top-down approaches. Controlled precipitation, evaporative precipitation into aqueous solution, and microprecipitation are examples of methods for producing API particles of reduced size by bottom-up approaches.

Solid dispersion technology is a widely implemented strategy for improving the dissolution properties and hence oral bioavailability of poorly water-soluble drugs. Solid dispersion technology is an approach to disperse a poorly soluble drug in a polymer matrix in the solid state. The drug can exist in amorphous or crystalline form in the mixture, which provides an increased dissolution rate and/or apparent solubility in the gastric and intestinal fluids. (see, e.g., A T M Serajuddin, J. Pharm. Sci. 88(10): 1058-1066 (1999) and M J Habib, Pharmaceutical Solid Dispersion Technology, Technomic Publishing Co., Inc. 2001). Several techniques have been developed to prepare solid dispersions, including co-precipitation (see, e.g., U.S. Pat. Nos. 5,985,326 and 6,350,786), fusion, spray-drying (see, e.g., U.S. Pat. No. 7,008,640), and hot-melt extrusion (see, e.g., U.S. Pat. No. 7,081,255). All these techniques provide a dispersed drug molecule in a polymer matrix, usually at the molecular level or in a microcrystalline phase. Solid dispersion systems provide increased wetable API surface area which significantly improves dissolution rates. Therefore, the absorption of these compounds can be improved by formulation as a solid dispersion system, if intestinal permeability is not the limiting factor, i.e. biopharmaceutical classification system (BCS) class 2 compounds (Amidon et al., 1995).

Many researchers have produced amorphous sold dispersion systems with various active compounds and polymeric carriers using hot-melt extrusion techniques to improve dissolution properties and bioavailability of poorly water-soluble drugs. Nakamichi et al. (U.S. Pat. No. 5,456,923), disclose a twin-screw extrusion process for producing solid dispersions of sparingly soluble drugs with various polymeric materials. Rosenberg and Breitenbach have produced solid solutions by melt extruding the active substance in a nonionic form together with a salt and a polymer, such as polyvinylpyrrolidone (PVP), vinylpyrrolidinone/vinylacetate (PVPVA) copolymer, or a hydroxyalkylcellulose (U.S. Pat. No. 5,741,519). Six et al., Brewster et al., Baert et al., and Verreck et al. have produced solid dispersions of itraconazole with improved dissolution rates by hot-melt extrusion with various polymeric carriers including hydroxypropylmethylcellulose, Eudragit E100, PVPVA, and a combination of Eudragit E100 and PVPVA (Pharmaceutical Research, 2003, 20(7): p. 1047-1054, Journal of Thermal Analysis and Calorimetry, 2002, 68: p. 591-601, Pharmaceutical Research, 2003. 20(1): p. 135-138, Journal of Pharmaceutical Sciences, 2004, 93(1): p. 124-131, International Journal of Pharmaceutics, 2003, 251(1-2): p. 165-174, WO2004004683, U.S. Pat. No. 6,509,038). Rambaldi et al. produced solid dispersions of itraconazole by hot-melt extrusion with hydroxypropyl-beta-cyclodextrin and hydroxypropylmethylcellulose for the improvement of aqueous solubility (Drug Development and Industrial Pharmacy, 2003, 29(6): p. 641-652). Verreck et al. produced solid dispersions of a water-insoluble microsomal triglyceride transfer protein inhibitor with improved bioavailability by hot-melt extrusion (Journal of Pharmaceutical Sciences, 2004. 93(5): p. 1217-1228). Hulsmann et al. produced solid dispersions of the poorly water soluble drug 17 beta-estradiol with increased dissolution rate by hot melt extrusion with polymeric carriers such as polyethylene glycol, PVP, and PVPVA along with various non-polymeric additives (European Journal of Pharmaceutics and Biopharmaceutics, 2000, 49(3): p. 237-242). Kothrade et al. demonstrated a method of producing solid dosage forms of active ingredients in a vinyllactam co-polymeric binder by hot-melt extrusion (U.S. Pat. No. 6,528,089). Grabowski et al. produced solid pharmaceutical preparations of actives in low-substituted hydroxypropyl cellulose using hot-melt extrusion techniques (U.S. Pat. No. 5,939,099). Breitenbach and Zettler produced solid spherical materials containing biologically active substances via hot-melt extrusion (International Pub. No. WO/2000/024382). Each of these systems differ from the present invention in that the API exists in the solid dispersion composition in a non-crystalline state. More specifically, the in situ conversion of the feed crystalline API to a non-crystalline form is not succeeded by a subsequent conversion back to a crystalline state.

Others have claimed hot-melt extruded compositions containing crystalline particles dispersed in a hydrophilic matrix. Ghebre-Sellassie, (International Pub. No. WO/1999/008660), discloses a method of producing crystalline solid dispersions of pharmaceutical agents in matrix of water-soluble polymers by hot melt extrusion at a temperature that softens, or even melts, the polymer but at which the drug remains crystalline. In this process, the mean particle diameter of the API in the crystalline solid dispersion is equivalent to that of the API in the process feed.

In contrast, according to the present invention, a crystalline solid dispersion in a water-soluble matrix is formed by first rendering the drug substantially non-crystalline and subsequently re-crystallizing it in-situ during the hot-melt extrusion process. The key advantage of the present invention is the ability to reduce the mean particle diameter of the API in the feed as it is dispersed in the polymer matrix. This is achieved by first destroying the API's crystalline structure (melting) and then recrystallizing it in a controlled manner to achieve a smaller mean particle diameter. The benefit of the claimed process is the ability to achieve faster dissolution rates than the particulate dispersions claimed by Ghebre-Sellassie (International Pub. No. WO/1999/008660 based on a reduction in particle size.

Miller et al., (U.S. Patent Application Publication No. 20080274194), claims a hot-melt extruded composition containing engineered drug particles dispersed in a hydrophilic polymeric matrix. The process of producing said compositions involves first the production of crystalline or amorphous engineered particles that are subsequently dispersed by hot-melt extrusion processing within a non-solubilizing polymeric carrier in such a way that the particle properties are not altered. By contrast, a particle preparation step is not included in the present invention. Rather, the benefit of particle engineering, i.e. particle size reduction, is achieved in situ during melt-extrusion processing. Also, Miller et al. describes a process in which the drug particles fed to the extrusion system are not altered during melt-extrusion processing, whereas by the present invention the drug particles fed to the extrusion system must first be altered (rendered non-crystalline) to achieve the desired product.

Thus, the present invention can be viewed as a hybrid technology; combining elements of bottom-up particle engineering with solid dispersion technology. Accordingly, the claimed process is distinctly unique from techniques described above. Through formulation design, equipment configuration, and process parameter optimization hot-melt extrusion technology is utilized to reduce the mean particle diameter of the crystalline API while simultaneously dispersing the API in a hydrophilic excipient matrix. The resultant crystalline solid dispersion yields faster dissolution rates of an API in a use environment with respect to other preparations containing the crystalline API (e.g. physical mixtures, co-micronized blends, etc.).

### SUMMARY OF THE INVENTION

The present invention provides a means of producing microparticles and nanoparticles of an API by shear induced controlled crystallization from a supercooled melt. In particular embodiments, the API is hydrophobic with a melting point less than 250° C. and a glass transition temperature below 45° C. The present invention can be classified as a bottom-up approach; i.e. the API particle assembly occurs from a molecular state. This would be opposed to a top-down approach where micro- and nanoparticles are formed by mechanical attrition; e.g. wet or dry milling. Bottom-up particle engineering techniques currently known in the art require the use of solvents which leads to a solvent removal and/or final drying step as part of the manufacturing process. The current invention circumvents the issue of solvent removal and secondary drying in that it is an anhydrous process in which particle formation is carried out from a molten state rather than a solution state.

The present invention also provides a method of producing crystalline solid dispersions of an API in a pharmaceutically acceptable carrier system. The present invention overcomes the drawbacks of the prior art with regard to crystalline solid dispersions produced by hot-melt extrusion techniques in that the present process provides a method of reducing the mean particle diameter of the API in situ while contemporaneously dispersing it in an excipient carrier. The resultant composition provides more rapid dissolution rates of the API in a use environment as compared to crystalline solid dispersions produced by hot-melt extrusion techniques previously disclosed in the art.

In certain embodiments, the present invention discloses crystalline solid dispersions of a CETP inhibitor in a hydrophilic excipient carrier system and a means for the preparation thereof. The present invention overcomes limitations of the prior art with regard to solid dispersions of certain CETP inhibitors. Some CETP inhibitors, e.g. dalcetrapib, are chemically and physically unstable in the amorphous state, and hence amorphous solid dispersions cannot be applied as a means of enhancing dissolution properties and oral absorption. The present invention provides a chemically and physically stable crystalline solid dispersion system of certain CETP inhibitors that produce rapid dissolution rates in a use environment.

In addition, a method is provided for forming a solid crystalline dispersion of an API by hot-melt extrusion processing. The process consists essentially of two operations: (1) melting of the API (and in some cases the excipient components) and (2) recrystallization of the API in the excipient matrix; carried out in series within the barrel of an extrusion system. First, the API is fed to a hot-melt extrusion system contemporaneously with the excipients composing the carrier system where they are conveyed through the extruder barrel by rotation of the screws. In the melting zone of the extruder barrel, the API and at least one of the excipients are rendered molten by heat exchange with the barrel walls with simultaneous mixing by the churning action of the screws. Subsequently, in the recrystallization zone of the extruder barrel, crystallization of the API is initiated by reducing the average temperature of the molten composite to below the melting point of the API through heat exchange with the chilled extruder barrels. This forces phase separation of the API from the excipient matrix and crystal seed (or nuclei) formation. Recrystallization of the API continues as the extruded material is conveyed through the crystallization zone where shear, imparted by proper screw design and rotation rate, acts to distribute crystal seeds throughout the molten bulk causing free drug molecules to more rapidly migrate to the surface of seeds. Once on the surface, molecules then become integrated into the seed lattice thereby growing the crystal.

The process is designed such that recrystallization of the API is carried out from the melt at a temperature below its melting point; i.e. a supercooled liquid state. In this supercooled state, viscosity is sufficiently high to restrict the growth of API crystals forming in the excipient matrix; however, not so high as to restrict mobility to the extent that amorphous or molecular API becomes frozen into the matrix and unable to crystallize. Balancing melt viscosity to achieve the desired crystallization is achieved through optimization of process parameters and formulation design.

Process design is critical to achieving the desired in situ crystallization and particle size reduction. The temperature profile in the extruder barrel must facilitate initial transformation of the API from the process feed to a non-crystalline state, e.g. melting; and then subsequently promote phase separation of the API from the excipient system to initiate the recrystallization process and control crystal growth thereafter. Screw design is also critical as shear must be applied in the melt zone of the barrel to facilitate melting of the API as well as downstream in the crystallization zone to accelerate the rate of recrystallization.

The excipient carrier consists essentially of one or more hydrophilic thermoplastic polymers: such as amonio methacrylate copolymer or polyoxyethylene-polyoxypropylene copolymer (poloxamer). This component of the carrier can be miscible with the API in the molten state as it has been determined that affinity between the drug and the polymer in the molten state tends to produce smaller API crystals in the final product. It is hypothesized that attractive interactions between the drug and polymer in the molten state slows the rate of phase separation upon transition into the crystallization zone of the process, thus restricting crystal seed size and increasing the number of discrete seed domains. Intuitively, it is understood that the number of crystal seeds is inversely correlated with mean crystal size in the final extruded product. Hence, it is apparent that carrier design is critical to controlling the particle size of the API in the crystalline solid dispersion claimed herein. The carrier may also contain functional excipients: such as, acidifying agents, wetting agents, surfactants, antioxidants, disintegrants, and the like.

Several compositions are provided comprising a cholesterol ester transfer protein inhibitor, a miscible hydrophilic thermoplastic polymer, and in some instances ancillary functional excipients. These compositions produce faster dissolution rates of CETP inhibitors in a use environment as compared to compositions containing crystalline CETP inhibitors produced by conventional means; e.g. co-micronization, wet-granulation, or the like.

In another aspect of the invention, different hydrophilic, thermoplastic polymers are disclosed. In one aspect of the invention, the polymer is amonio methacrylate copolymer and in another aspect of the invention the polymer is polyoxyethylene-polyoxypropylene copolymer (poloxamer).

In another aspect of the invention, certain ancillary functional excipients improve product performance. For example, mannitol and isomalt serve as water soluble diluents acting as dissolution aids. In addition, polyoxyethylene-polyoxypropylene copolymer acts as a crystallization inducing agent; imparting positive influence on product stability.

The various aspects of the present invention each provide one or more of the following advantages. The process of the present invention provides a means of producing nanoparticles and/or microparticles of an API by a bottom-up approach without the use of solvents and by continuous processing. The process of the present invention provides a means of producing solid microcrystalline and/or nanocrystalline dispersions of an API in a hydrophilic matrix by hot-melt extrusion processing without the need for preprocessing of the API, e.g. milling to achieve the desired particle size. For example, the compositions of the present invention can improve the dissolution rate of certain CETP inhibitors in a use environment as compared to compositions containing crystalline CETP inhibitors produced by conventional means; e.g. co-micronization, wet granulation, or the like. Such dissolution rate enhancements are unexpectedly large relative to that of typical crystalline formulations of CETP inhibitors (i.e., reaching 100% dissolved in 10 minutes in some cases as compared to 30% dissolved for control formulations in an in vitro test solution). Owing to the insolubility of some CETP inhibitors, such a large dissolution rate enhancement is necessary for oral administration in order to render convenient dose amounts therapeutically effective.

### BRIEF DESCRIPTION OF THE DRAWINGS

FIG. 1 provides a schematic representation of the claimed hot-melt extrusion process.

FIG. 2 provides an explanation of the screw element type used in tables 3, 6, and 9. Note that the unit for length is in millimeters (mm).

FIG. 3 shows the x-ray diffraction pattern of a composition produced according to Example 1 in comparison to bulk dalcetrapib.

FIG. 4 shows the particle size analysis report for bulk dalcetrapib.

FIG. 5 shows the particle size analysis report for dalcetrapib contained in the matrix of the composition described in Example 1.

FIG. 6 reflects the comparative dissolution performance of: (1) a nanoparticle suspension of dalcetrapib produced by wet-milling (shown as triangles), (2) the hot-melt extruded granules produced according to Example 1 (shown as diamonds), and (3) micronized dalcetrapib produced by jet-milling (shown as squares).

FIG. 7 shows a representative x-ray diffraction pattern of the compositions produced according to Example 5 in comparison to bulk dalcetrapib.

FIG. 8 provides the particle size analysis report for dalcetrapib contained in the matrix of the composition described in Example 5 containing 60% (w/w) dalcetrapib.

FIG. 9 provides the particle size analysis report for dalcetrapib contained in the matrix of the composition described in Example 5 containing 70% (w/w) dalcetrapib.

FIG. 10 reflects the comparative dissolution performance of: (1) the hot-melt extruded granules produced according to Example 5 with 60% (w/w) dalcetrapib (shown as squares) (2) the hot-melt extruded granules produced according to Example 5 with 70% (w/w) dalcetrapib (shown as diamonds).

FIG. 11 reflects the comparative dissolution performance of: (1) the hot-melt extruded granules produced according to Example 9 with 60% (w/w) dalcetrapib (shown as squares) (2) the hot-melt extruded granules produced according to Example 9 with 70% (w/w) dalcetrapib (shown as triangles).

### DETAILED DESCRIPTION OF THE INVENTION

Unless otherwise indicated, the following specific terms and phrases used in the description and claims are defined as follows:

The term “use environment” refers to an environment where the pharmaceutical compositions of the present invention are normally used including the in vivo environment of the gastrointestinal (GI) tract of a mammal, particularly a human, and the in vitro environment of a test solution, such as simulated gastric fluid (SGF), phosphate buffered saline (PBS), or a derivative of simulated intestinal fluid (SIF).

The term “CETP inhibitor” refers to a cholesteryl ester transfer protein inhibitor such as (but not limited to) dalcetrapib.

The term “API” refers to an active pharmaceutical ingredient including (but not limited to) CETP inhibitors such as dalcetrapib.

The term “amino methacrylate copolymer” refers to a polymerized copolymer of (2-dimethylaminoethyl)methacrylate, butyl methacrylate, and methyl methacrylate which has a mean relative molecular mass of about 150,000. The ratio of (2-dimethylaminoethyl)methacrylate groups to butyl methacrylate and methyl methacrylate groups is about 2:1:1. In addition, the copolymer contains not less than 20.8 percent and not more than 25.5 percent of dimethylaminoethyl groups, calculated on a dried basis.

The term “isomalt” refers to the disaccharide of 1-O-alpha-D-Glucopyranosyl-D-mannitol.

The term “poloxamer” refers to a nonionic triblock copolymer composed of a central hydrophobic chain of polyoxypropylene (poly(propyleneoxide)) flanked by two hydrophilic chains of polyoxyethylene (poly(ethyleneoxide)). Poloxamers can be referred to by the letter “P” (for poloxamer) followed by three digits wherein the first two digits multiplied by 100 gives the approximate molecular mass of the polyoxypropylene core, and the last digit multiplied by 10 gives the percentage of polyoxyethylene content (e.g., P407=poloxamer with a polyoxypropylene molecular mass of 4,000 g/mol and a 70% polyoxyethylene content).

The term “therapeutically effective amount” means an amount of an API that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. The daily dosage can be administered as a single dose or in divided doses, or for parenteral administration, it may be given as continuous infusion.

The term “pharmaceutically acceptable carrier” or “excipient carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

In detail, the present invention relates to a composition and method of reducing the mean particle diameter of an API while simultaneously dispersing the crystalline API particles in an excipient carrier by hot-melt extrusion processing. In particular embodiments, the API is hydrophobic with a melting point less than 250° C. and a glass transition temperature below 45° C. Examples of such APIs include dalcetrapib, ibuprofen, ketoprofen, indomethacin, and acetaminophen. The method of the present invention can be described as a bottom-up particle formation technique in that microparticles and nanoparticles are assembled from a molecular state. The method utilizes traditional screw extrusion equipment to generate a supercooled molten form of the API with excipients, then imparts shear onto this supercooled system to accelerate crystallization of the API. The particle size of the recrystallizing API is controlled by the extrusion process parameters and carrier formulation.

The invention also concerns a composition comprising cholesteryl ester transfer protein (CETP) inhibitors dispersed as crystalline microparticles and/or nanoparticles in a hydrophilic pharmaceutically acceptable excipient carrier and a method of producing said composition. Cholesteryl ester transfer protein (CETP) inhibitors elevate certain plasma lipid levels, including high density lipoprotein (HDL)-cholesterol and lower certain other plasma lipid levels, such as low density lipoprotein (LDL)-cholesterol and triglycerides and accordingly treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans. Thus, CETP inhibitors should result in higher HDL cholesterol levels and lower LDL cholesterol levels. To be effective, such CETP inhibitors must be absorbed into the blood. Oral dosing of CETP inhibitors is preferred because to be effective such CETP inhibitors must be taken on a regular basis, such as daily. Therefore, it is preferred that patients be able to take CETP inhibitors by oral dosing rather than by injection.

CETP inhibitors, particularly those that have high binding activity, are generally hydrophobic, have extremely low aqueous solubility and have low oral bioavailability when dosed conventionally. Such compounds have generally proven to be difficult to formulate for oral administration such that high bioavailabilities are achieved. For example, CETP inhibitors generally have (1) extremely low solubilities in aqueous solution (i.e., less than about 10 μg/mL) at physiologically relevant pH (e.g., any pH of from 1 through 8) measured at about 22° C.; (2) a relatively hydrophobic nature; and (3) a relatively low bioavailability when orally dosed in the crystalline state. Indeed, the solubility of some CETP inhibitors is so low that it is in fact difficult to measure. Accordingly, when CETP inhibitors are dosed orally, concentrations of CETP inhibitors in the aqueous environment of the gastrointestinal tract tend to be extremely low, resulting in poor absorption from the GI tract to blood. The hydrophobicity of CETP inhibitors not only leads to low equilibrium aqueous solubility but also tends to make the drugs poorly wetting and slow to dissolve, further reducing their tendency to dissolve and be absorbed from the gastrointestinal tract. This combination of characteristics has generally resulted in the bioavailability for orally dosed conventional crystalline or amorphous forms of CETP inhibitors to be quite low, often having absolute bioavailabilities of less than 1%. Thus, it has proven to be difficult to formulate certain CETP inhibitors for oral administration such that therapeutic blood levels are achieved.

Accordingly, CETP inhibitors require some kind of modification or formulation to enhance their solubility and thereby achieve good bioavailability. Surprisingly, the compositions of the present invention provide unusually rapid dissolution rates in an aqueous environment of use compared with other conventional crystalline compositions used to formulate poorly soluble, hydrophobic drugs. The inventors of the present invention have found a new method for reducing the particle size of certain CETP inhibitor crystals while simultaneously dispersing them in a hydrophilic carrier. Preparing CETP inhibitors as compositions comprising a crystalline solid dispersion by this method improves the aqueous dissolution rate of the CETP inhibitors. Thus, the invention provides more rapid dissolution of certain CETP inhibitors in a use environment than compositions containing crystalline CETP inhibitors produced by conventional means; e.g. co-micronization, wet-granulation, and the like.

With the goal of improving the oral bioavailability of CETP inhibitors, Curatolo et. al. (U.S. Pat. No. 7,115,279 and U.S. Patent Application No. 20060211654) and Crew et al. (U.S. Pat. No. 7,235,259 and U.S. Patent Application Publication Nos. 20070282009 and 20030186952), disclose amorphous solid dispersions of CETP inhibitors with a concentration enhancing polymer for improved oral bioavailability. Among other processes, the inventors claim hot-melt extrusion as a means of producing said compositions. This approach differs from the present invention in that the resultant product contains the CETP inhibitor in an amorphous state, which is in contrast with the significantly crystalline hot-melt extruded composition claimed herein. Owing to the chemical instability of some CETP inhibitors in the amorphous state, this amorphous solid dispersion formulation approach is not practical. It was precisely this amorphous chemical instability that necessitated the present invention in which modified dissolution and improved bioavailability of the CETP inhibitor is achieved from the crystalline form of the API by reducing particle size and embedding the API in a hydrophilic excipient matrix. Additionally, the term concentration enhancing polymer, as used by Curatolo et al. and Crew et al., appears in the pharmaceutical literature to describe a polymeric excipient which enhances the supersaturated state of a therapeutic compound in an aqueous use environment. In contrast, the present invention seeks only to improve the dissolution rate of CETP inhibitors in a crystalline state rather than generating supersaturation from an amorphous state.

### Method of Preparation

A method is provided for reducing the mean crystalline particle diameter of an API while simultaneously dispersing the particles in an excipient carrier by hot-melt extrusion processing. The process can be generally regarded as a bottom-up particle engineering technique and the resultant composition can be regarded as a crystalline solid dispersion.

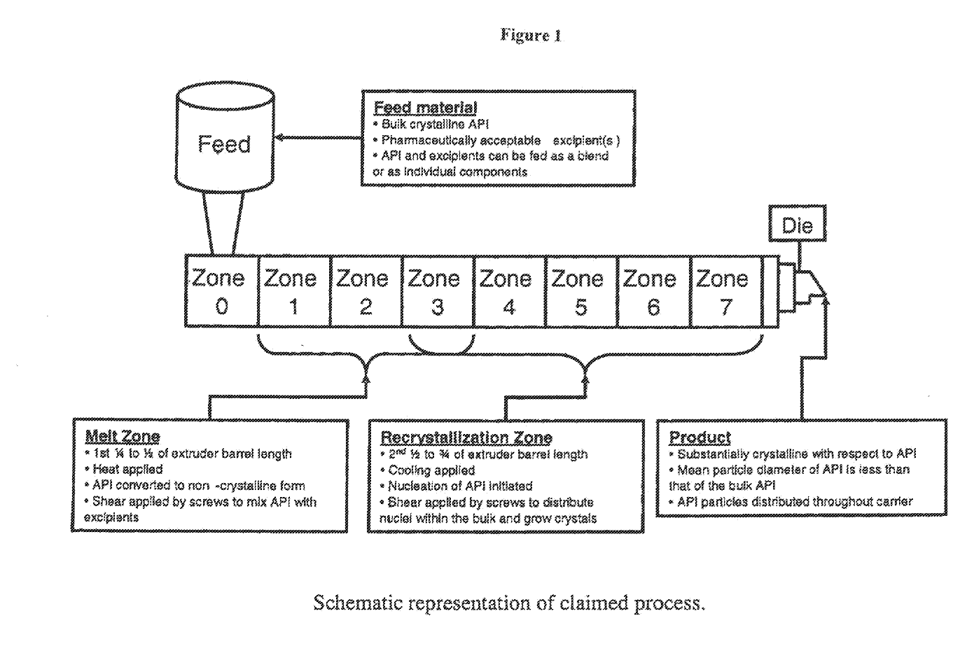

The process consists essentially of two operations: (1) generating a supercooled liquid state of the API in the presence of excipients and (2) forcing extensive crystallization of the API from the supercooled system. By the present invention, these operations are carried out in series within a typical melt extrusion system. The API is fed to a hot-melt extrusion system contemporaneously with the excipients comprising the carrier system where they are conveyed through the extruder barrel by rotation of the screws. In what shall be referred to as the melting zone of the extruder barrel, the API and at least one of the excipients are rendered molten by heat exchange with the barrel walls with simultaneous mixing by the churning action of the screws. Subsequently, in what shall be referred to as the recrystallization zone of the extruder barrel, crystallization of the API is initiated by reducing the temperature of the molten composite to below the melting point of the API by way of heat exchange with the chilled extruder barrels. This forces phase separation of the API from the excipient matrix and crystal seed (or nuclei) formation. Recrystallization of the API continues as the extruded material is conveyed through the crystallization zone where shear, imparted by proper screw design and rotation rate, acts to distribute crystal seeds throughout the molten composite causing free drug molecules to adhere to the surface of expanding seeds, growing crystals presumably by an Ostwald ripening process. FIG. 1 provides a schematic description of the process.

### Description of Process

### Feeding

The API and the excipients comprising the carrier system can be pre-blended and fed to the extrusion system as a single powder mass, or alternatively each component can be fed individually. Feed materials can be fed to the extrusion system using a twin screw gravimetric feed system, single screw agar, or the like.

### Melting

After feeding the powder components into the barrel of the extrusion system, the next step in the process converts the API into a liquid state. To this end, in the first one-fourth to one-half of the barrel length, heat exchange occurs at the barrel walls to increase the average temperature of the API/excipient mixture near or beyond the melting point of the API. Alternatively, the set-temperatures of the barrels in the melt zone could be set at a temperature below the melting point of the API, but near or beyond the melting point of one or more excipients. In this case, the molten excipient(s) would act to solubilize the API and convert crystalline particles into a liquid state. Either way, the bulk crystalline API must be rendered molten in the melting zone of the extruder barrel; this can be accomplished by either heating the API near or beyond its melting point or dissolving the drug into a molten excipient.

Screw design in the melt zone of the extrusion system is also important. The screw should be configured with sufficient dispersive and/or distributive mixing elements in the melt zone to enable intimate mixing of the API/excipient system once rendered molten. The geometries of these mixing elements is not critical. Any standard dispersive or distributive mixing elements commonly used in twin screw extrusion systems will suffice; so long as sufficient mixing is applied at a point in the process where the feed material is rendered sufficiently molten. Homogenous distribution and intimate contact of the API with the carrier is crucial to controlling crystallization in the recrystallization zone of the extruder system and achieving very fine crystalline API particles.

### Recrystallization

After the API has been converted to a liquid state and intimately mixed with the excipient(s), it is then conveyed by the action of the rotating screws into the crystallization zone. The set temperatures of the extruder barrels in the recrystallization zone are below the melting point of the API. Heat exchange occurs at the barrel walls to cool the molten composite exiting the melt zone to reduce the temperature of the composite below the melting temperature of the API. It is at this transition point that a supercooled liquid state of the API is generated. From this supercooled state, the API is able to crystallize with the viscosity of the supercooled system providing sufficient retardation of crystal growth to allow for particle size control. However, melt viscosity should not be so great that amorphous API becomes frozen in the excipient matrix and unable to crystallize (i.e., greater than 10,000 Pa·s as measured by a shear stress controlled rotational rheometer at 10 rad/s at a temperature close to that of the process).

The carrier system of the invention contains at least one excipient which is miscible with the API in a liquid or molten state at a temperature near the melting point or glass transition temperature (T_{g}) of the API or excipient. However, this mixture becomes increasingly less miscible as the temperature of the system is reduced below the melting point/T_{g} of the API and/or excipient and ultimately to room temperature. The solid state (at ambient conditions) solubility of the API and the excipient should ideally be negligible in order to produce an entirely crystalline composite with respect to the API.

Miscibility in the molten state yields a molecularly disperse system (with respect to the API) at the transition point from the melt zone. This implies that API molecules are homogenously dispersed within the melt and spatially separated by excipients. Further, there are no discernable API-rich domains in the melt, or these domains are extremely small in size; i.e. <100 nm. As the melt transitions into the recrystallization zone and becomes supercooled, the API will begin to phase separate from the excipient forming the nuclei that will later grow to become the API crystals. A homogenous distribution of molecularly disperse API in the excipient matrix will ensure that nuclei formation is vast within the excipient network and contained within the immediate environment by the excipient network. The formation of numerous nuclei, each shrouded by the excipient network, creates numerous points of crystal growth and prevents particle coalescence. It is intuitively understood that a greater number of growth points results in a smaller crystal particle size. Therefore, it can be understood that API-excipient miscibility as it relates to preventing macroscopic phase separation prior to nuclei formation is critical to controlling crystalline particle size in the final extruded product.

To further illustrate the concept, the converse can be considered. Limited miscibility between the API and the excipient system will lead to large-scale phase separation of the melt in the transition to the recrystallization zone. The result will be API globule formation, growth of crystal seeds without steric interference by the excipients, and ultimately larger crystals in the final product. On the other extreme, if the API and the excipient system were highly miscible below the melting point of the API, phase separation would not occur and a composition with a significant amorphous fraction would result. Such a composition would not be suitable for APIs that are not chemically stable in the amorphous state, such as dalcetrapib. Hence, the novelty of this invention as applied to a compound such as dalcetrapib is the achievement of an increased number of very fine crystals resulting in a more rapid dissolution rate from a chemically stable crystalline composition.

Once crystallization has been initiated by the formation of nuclei, the next step in the crystallization process is to grow crystals from seeds up to the point that free API molecules in the composite have been exhausted and complete crystallization is achieved. The presumed model for crystal growth is surface deposition of free molecular API to the surface of a propagating crystal as per an Oswalt ripening process. In a static system, this process can require a significant amount of time to complete as transport of molecules to a crystal surface is primarily diffusion controlled. From a molten state, the process would proceed particularly slow as viscosity can be a substantial limitation to diffusion.

However, in practicing the present invention, the crystallization process is carried out in a matter of seconds by inducing shear within the supercooled molten composite. This is achieved by the design of the extrusion screw system. Distributive and/or dispersive mixing elements are placed between the midpoint and end of the recrystallization zone and the rotational motion of the screws at this point acts to aggressively mix the nuclei-rich supercooled system. Again, the geometries and sizes of these mixing elements is not critical. Any standard dispersive or distributive mixing elements commonly used in twin screw extrusion systems will suffice; so long as sufficient mixing is applied at the point in the process deemed optimal for crystallization. The shear imparted on the system by the rotation of the kneading screw elements distributes nuclei within the bulk, increasing the collision frequency of free molecular API with a crystal seed surface and consequently accelerating crystal growth. Within the crystallization zone, kneading elements are present in segments, interspaced by conveying elements to allow for continued crystal growth as the material is conveyed through the barrel. By continued cooling of the system and the application of shear, the API continues to phase separate from the excipient system in the formation of crystals up to the point that free molecular API is exhausted and complete crystallization is achieved.

### Finishing

Upon exiting the extruder barrel, the extrudate is collected by a suitable takeoff system, such as: a conveyor belt, a roller, in-line pelletizer, or the like. The takeoff equipment is typically equipped with cooling capabilities, i.e. air jets or circulating liquid coolant, which can further cool the extrudate and complete the recrystallization process. The material collected by the take off system can be in the form of strands, films, flakes, pellets, granules, or the like. Regardless of the final shape, each embodiment of the final extrudate product is comprised of crystalline API (with a mean particle diameter less than that of the starting bulk API material) dispersed in the excipient carrier system. The collected hot-melt extruded product can then be milled into a fine granulate suitable for further processing into a final dosage form; e.g. tablet, capsule, sachet, powder for constitution, or the like.

### Compositions of CETP Inhibitors and Hydrophilic Carriers

### Miscible Carrier Excipient

Critical to controlling crystallization of an API from the melt is selection of co-processed excipients. It is preferred that at least one of the excipients be miscible with the API in the molten state (i.e., amino methacrylate copolymer, poloxamer 188, and poloxamer 407 are miscible with dalcetrapib). This ensures that a molecular mixture of the API is generated with at least one of the excipient carriers in the above described melt zone of the extruder. Generating a molecular mix ensures that large scale phase separation of the molten API from the excipient system does not occur; this would be analogous to oiling out with regard to crystallization from solution. Stearic hindrance of API crystal growth by the excipient system in the melt is the underlying principal of controlling crystal growth by the present invention. If large scale phase separation occurs in the melt, there will be no physical interruption of the crystal growth process and hence limited control of crystal size.

### Immiscible Carrier Excipient

In some applications of the present invention it may also be advantageous to incorporate an excipient in the carrier system which is immiscible with the API in addition to a miscible excipient carrier. The purpose of this immiscible excipient is to function as an anti-solvent and expel residual molecular API from the excipient system which would otherwise remain in “solution” based on thermodynamic solubility with the miscible excipient(s). This is particularly advantageous when the API is chemically unstable in the amorphous form.

### Ancillary Excipients

In addition to the above described excipients, additional functional excipients may be required to improve performance with respect to stability, dissolution, or downstream processing. These excipients could include anti-oxidants, disintegrants, flow aids, compression aids, lubricants, and the like.

### EXAMPLES

### Example 1

### Production of a Crystalline Solid Dispersion of Dalcetrapib in Amino Methacrylate Copolymer

### Process Steps

### Feeding

The API and the excipients comprising the carrier system can be pre-blended and fed to the extrusion system as a single powder mass, or alternatively each component can be fed individually. In this case, the API and excipient components, in the ratio provided in the table below, are first pre-blended in a suitable powder blender (bin or twin-shell).

TABLE 1
Composition

| Component | % (w/w) |
|---|---|
| Dalcetrapib | 70.0 |
| Amino methacrylate copolymer USP/NF | 29.75 |
| Fumed silica | 0.25 |

Table 1 provides a quantitative composition of a crystalline solid dispersion of dalcetrapib in a matrix consisting essentially of amino methacrylate copolymer.

### Hot-Melt Extrusion

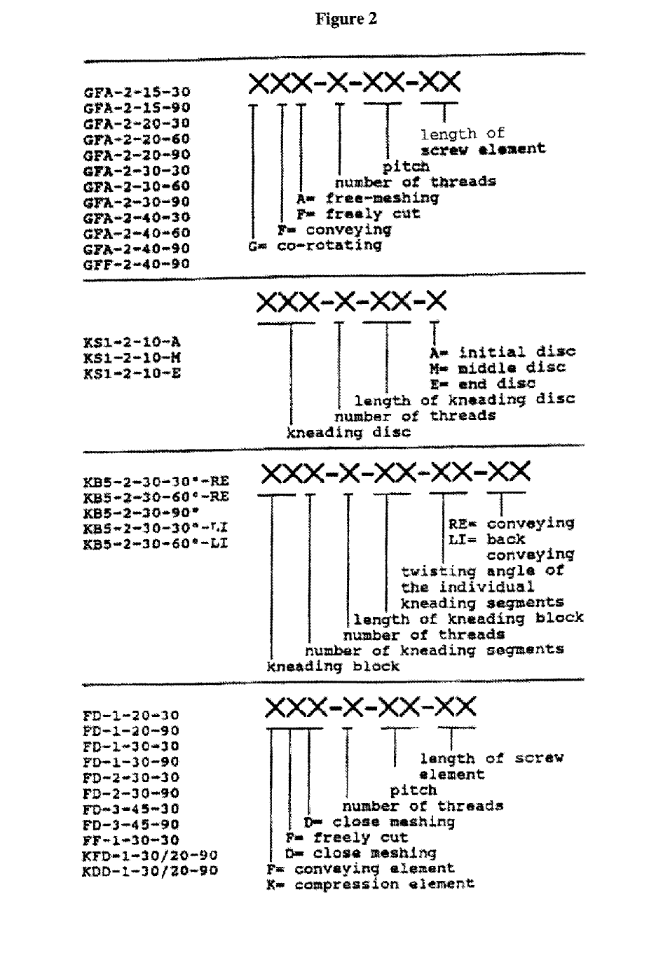

The resulting powder from blending is then fed into a commonly used twin-screw extrusion system (American Leistritz model Micro-18 lab twin-screw extruder) using a common loss on weight feeder operated at a rate of 20 g/min. The barrel temperature profile (for each zone as shown in FIG. 1) and screw configuration are provided below. The screw element nomenclature is explained in FIG. 2.

TABLE 2

| Barrel location | Feed | 1 | 2 | 3 | 4 | 5 | 6 | 7 | Die |
|---|---|---|---|---|---|---|---|---|---|
| Temperature | N/A | 65 | 65 | 65 | 65 | 15 | 15 | 15 | 25 |
| Set Point (° C.) | | | | | | | | | |

Table 2 depicts the temperature at successive locations along the barrel of a twin screw extrusion system used to process the composition provided in Table 1.

TABLE 3

| | Screw Element | Element |
|---|---|---|
| Barrel location | Type | Number |
| Feed | GFA-2-30-90 | 1 |
| | GFA-2-20-60 | 2 |
| | KB4-2-20-30° | 3 |
| | KB4-2-20-60° | 4 |
| | GFA-2-20-30 | 5 |
| | KB4-2-20-90° | 6 |
| | GFA-2-30-90 | 7 |
| | KB 4-2-20-30° | 8 |
| | KB4-2-20-60° | 9 |
| | KB4-2-20-60° | 10 |
| | KB4-2-20-90° | 11 |
| | GFA-2-20-30 | 12 |
| | KB 4-2-20-30° | 13 |
| | KB4-2-20-60° | 14 |
| | KB4-2-20-60° | 15 |
| | GFA-2-20-30 | 16 |
| | KB 4-2-20-30° | 17 |
| | KB4-2-20-90° | 18 |
| | GFA-2-30-90 | 19 |
| | GFA-2-20-60 | 20 |
| Exit | Die | NA |

Table 3 provides the screw element type at successive locations along the barrel of the twin screw extrusion system used to process the composition provided in Table 1 (i.e. beginning at the feed extending to the barrel exit).
FIG. 2 provides an explanation of screw element type terminology.

The temperature set points in barrel locations one through four are set to the melting point of dalcetrapib to ensure that within this region of the barrel the crystalline API is melted, i.e. converted to a liquid state. Within this region, kneading elements (element numbers 3, 4, 6, 8, 10, and 11 are incorporated into the screw design to promote melting of the API and thorough mixing with the molten polymer. Dalcetrapib and amino methacrylate copolymer (butyl methacrylate/2-dimethylaminoethyl methacrylate copolymer) are completely miscible at a 70:30 ratio at 65° C. Miscibility of the API and the polymer ensures molecular mixing which is critical to controlling dalcetrapib crystallization in the subsequent “crystallization region” of the extruder barrel.

The temperature set points are 15° C. at barrel blocks five through seven for the purpose of shock-cooling the molten composite. Rapid cooling in this fashion promotes sudden phase separation of dalcetrapib from the molten polymer. Sudden phase separation promotes the formation of numerous dalcetrapib crystal nuclei which are the seeds for crystal growth. Considering that the reservoir of free dalcetrapib molecules is finite, it is understood that as the number of seeds increases with which free molecules can adhere to during the crystallization process, the size of the crystals formed at the point where the free molecules are exhausted correspondingly decrease. Therefore, shock cooling in this manner to promote extensive seed formation is essential to achieving fine particles of crystalline dalcetrapib.

The kneading elements incorporated into the screw design at the crystallization region of the extruder barrel (i.e., element numbers 14, 15, 17 and 18 in table 3) act to shear the semi-molten composite via rotation of the screw which provides the mixing function necessary to disperse dalcetrapib crystal seeds throughout the bulk fluid and accelerate crystal formation. By this mixing action of the screw extrusion system the crystallization process is able to be completed on the order of minutes. Conversely, crystallization of dalcetrapib from a stagnant super-cooled melt would require on the order of hours to complete.

### Product Collection

At the exit of the barrel through the die, crystallization of dalcetrapib is near complete and consequently the extrudate is a solid mass which can be easily handled by typical equipment designed to take-off extruded products. In this case, the extrudate is transported from the die exit by a typical belt conveyor to an in-line pelletizer (BT-25 Strand Pelletizer, Bay Plastics Machinery). Depending on the application, the pellets can then be milled using a standard hammer mill and incorporated into a blend for encapsulation, tableting, etc.

### Example 2

### X-Ray Diffraction Analysis of a Crystalline Solid Dispersion of Dalcetrapib in Amino Methacrylate Copolymer

X-ray diffraction (XRD) analysis was performed on bulk dalcetrapib and the composition produced according to Example 1 to confirm the crystallinity and polymorph of the API following the HME process.

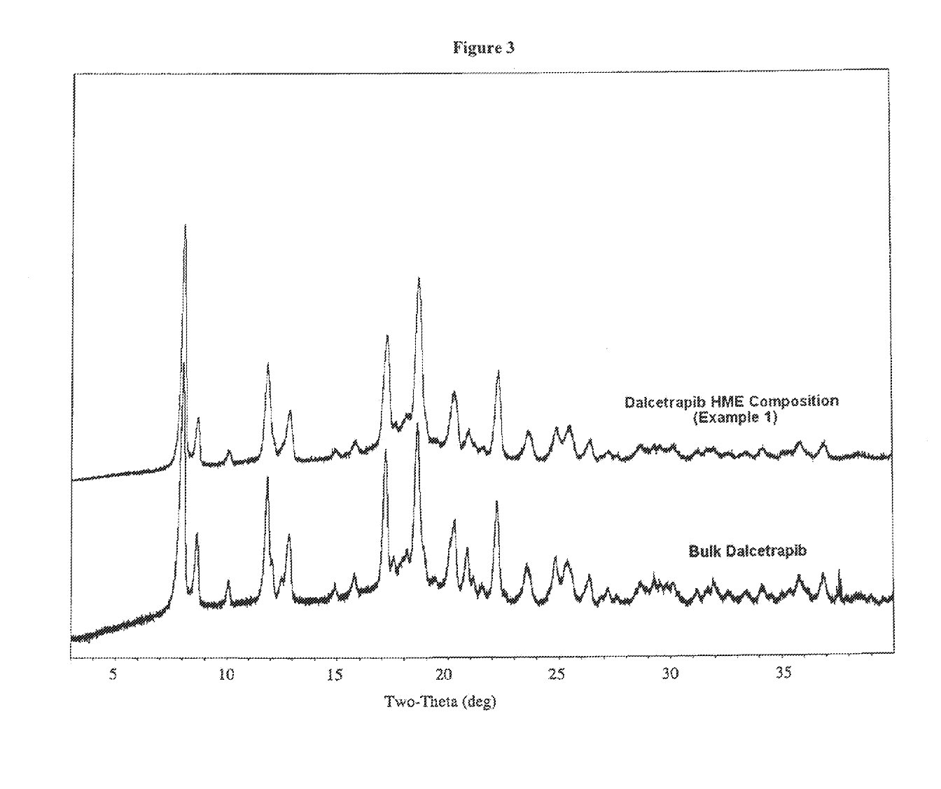

XRD analysis was performed using a Bruker D8 XRD Model D8 Advance x-ray diffractometer. Powder samples were smoothly packed into an aluminum sample holder and loaded onto the sample stage for analysis. The results of this analysis are presented in FIG. 3 where it is seen that the composition produced according to Example 1 exhibits an x-ray diffraction pattern very similar to that of bulk dalcetrapib. This indicates that dalcetrapib contained in the composition produced according to Example 1 is substantially crystalline and the crystalline polymorph is identical to that of the bulk API. Thus, it is demonstrated that dalcetrapib is completely recrystallized by the extrusion process following the initial melt transition, and the final crystalline form is identical to that of the bulk API.

### Example 3

### Particle Size Analysis of a Crystalline Solid Dispersion of Dalcetrapib in Amino Methacrylate Copolymer

The particle size distribution of dalcetrapib crystals from the bulk API and in the matrix of a hot-melt extruded composition produced according to Example 1 was determined according to the following method:

A Malvern MasterSizer 2000 was used for particle size measurement. The Fraunhofer optical model employed for analysis. The sample handling unit was a Hydro 2000S sonicator: Elma Model 9331. Sample measurement time was 20,000 snaps. The sample background time was 20,000 snaps. The dispersant media was 0.1N HCl, and the pump/stir speed was 2000 RPM.

Sample preparation was as follows: About 10-15 mg of the sample was weighed in 20 mL scintillation vial and 10 mL of de-ionized 0.1N HCl was added. The sample was vortexed for 15 seconds and then sonicated for 10 minutes @ 100% power.

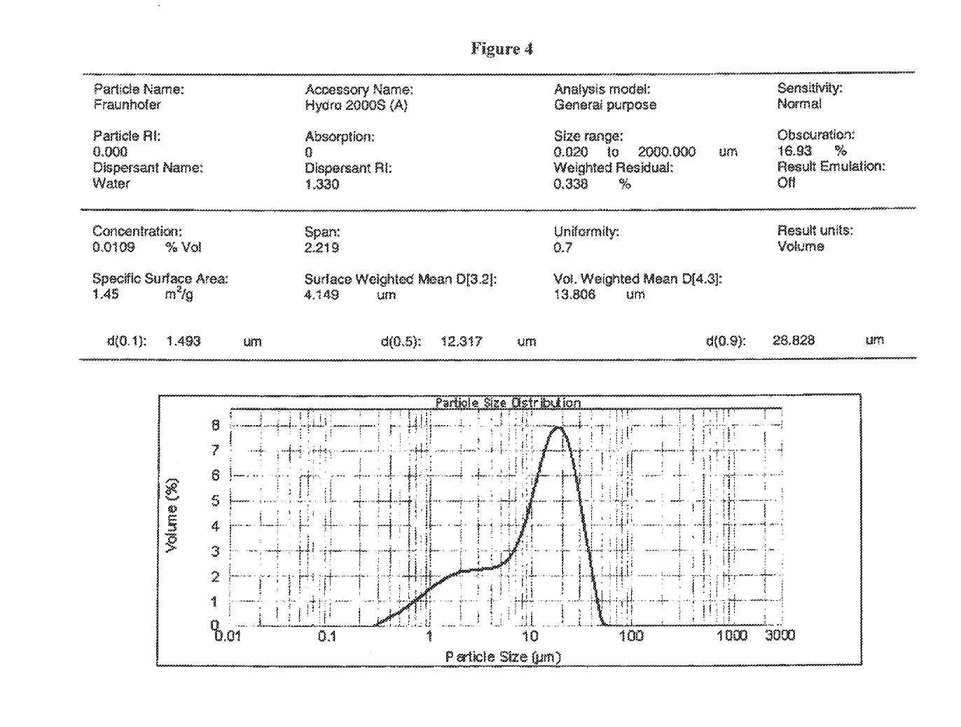

As is shown in FIG. 4, the mean particle diameter D(0.1), D(0.5), and D(0.9) values for bulk dalcetrapib are 1.493, 12.317, and 28.828 μm respectively. The mean D(0.1), D(0.5). and D(0.9) values for The HME composition produced according to Example 1 are 0.617, 1.386, and 3.320 μm respectively. Taking the D(0.5) value as an average particle size, the HME process described herein was able to reduce the particle size of bulk dalcetrapib by nine-fold. This example thus illustrates a novel aspect of the present invention in that significant primary particle size reduction is achieved during the process with simultaneous dispersion in the polymer matrix.

### Example 4

### Comparative Dissolution Analysis of a Crystalline Solid Dispersion of Dalcetrapib in Amino Methacrylate Copolymer Versus a Dalcetrapib Nanoparticle Suspension and Micronized Dalcetrapib

Dissolution analysis of the dalcetrapib HME composition produced according to Example 1 and control formulations was conducted by the following method

USP Apparatus II (paddle) dissolution testing was conducted using a Distek Evolution 6300 dissolution tester (Distek Inc., North Brunswick, N.J., USA) at a paddle speed of 75 RPM. The dissolution media was 1000 mL of 0.1 N HCl containing 0.75% HTAB (hexadecyltrimethylammonium bromide) equilibrated at 37°±0.5° C. Six replicate samples equivalent to 300 mg dalcetrapib were tested simultaneously. The mean concentration value of these six samples was calculated and reported for each time point. Sample concentrations were determined using an online fiber optic UV detection at 248 nm (Rainbow Dynamic Dissolution Monitor System, Delphian Technology, Woburn, Mass., USA).

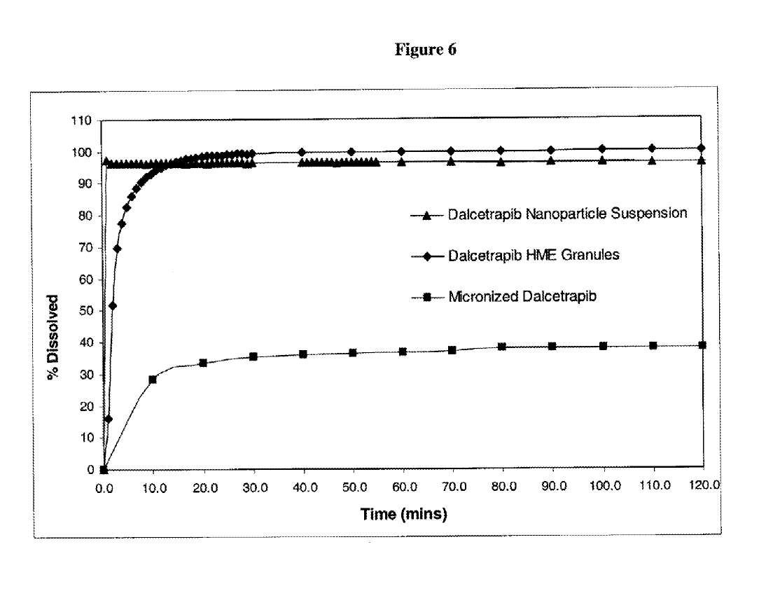

Dissolution analysis of the HME composition produced according to Example 1 was conducted in comparison with a nanosuspension of dalcetrapib (D(0.5)=300 nm) produced by standard wet milling techniques and micronized dalcetrapib (D(0.5)=2.3 μm) produced by conventional jet milling. The results of this analysis are presented in FIG. 6 which shows that the nanosuspension of dalcetrapib dissolved instantly reaching 100% in about approximately one minute. The HME granules (milled extrudate) described in Example 1 also exhibit an exceptionally rapid dissolution profile, achieving 100% dissolved in approximately ten minutes. Micronized dalcetrapib exhibited a much slower and less extensive dissolution profile achieving only 30% dissolved in the first ten minutes and increasing only slightly after two hours of dissolution testing.

The near instant dissolution of the nanosuspension is expected due to the extensive surface area that is created when the size of the crystalline particles is reduced to 300 nm. However, the rapid dissolution of the HME granules is unexpected in that the crystalline dalcetrapib particles contained in the matrix are approximately five-fold larger than the nanosuspension and approximately equal to the micronized dalcetrapib which showed quite slow and limited dissolution. Therefore, the rapid dissolution profile of the HME granules can only be partially attributed to the particle size reduction of the dalcetrapib crystals during the extrusion process. It is believed the primary contributing factor toward the rapid dissolution of the HME granules is the intimate mixing of the drug particles and the polymer achieved during the process of the present invention. For conventional crystalline solid dispersions produced by melt extrusion in which a phase change of the API does not occur, intimate mixing is limited to surface coverage of the particles by the polymer. Surface coverage is also achieved by the current process which improves the wetability of the drug particles in the matrix and contributes to the rapid dissolution profile of the HME granules. However, a unique attribute of the current invention is that the drug-polymer interactions extend beyond surface interactions. It is believed that when recrystallizing the drug in the presence of the molten polymer, the polymer molecules become partially incorporated into the crystal lattices of the drug particles. In essence this creates de facto crystal defects that reduce the stability of the crystal lattices (increase free energy) thereby reducing the energy input required to break apart the particles during the dissolution process. This would explain the significantly more rapid dissolution profile of the HME granules versus the micronized dalcetrapib and similarity to the nanosuspension despite a significantly greater mean particle diameter.

### Example 5

### Production of Crystalline Solid Dispersions of Dalcetrapib of 60% and 70% (w/w) Drug Loading in a Poloxamer 188/D-Mannitol Matrix

### Process Steps

### Feeding

The API and the excipients comprising the carrier system can be pre-blended and fed to the extrusion system as a single powder mass, or alternatively each component can be fed individually. In this case, the API and excipient components, in the ratios provided in the table below, are first pre-blended in a suitable powder blender (bin or twin-shell).

TABLE 4
Compositions

| Component | % (w/w) | % (w/w) |
|---|---|---|
| Dalcetrapib | 60.0 | 70.0 |
| Poloxamer 188 | 25.0 | 20.0 |
| D-mannitol | 15.0 | 10.0 |

Table 4 provides quantitative compositions of a crystalline solid dispersion of dalcetrapib with 60% and 70% (w/w) drug loading in a matrix consisting essentially of poloxamer 188 and D-mannitol.

### Hot-Melt Extrusion

The resulting powder from blending is then fed into a commonly used twin-screw extrusion system (American Leistritz model Micro-18 lab twin-screw extruder) using a common loss on weight feeder operated at a rate of 20 g/min. The barrel temperature profile (for each zone as shown in FIG. 1) and screw configuration are provided below.

TABLE 5

| Barrel location | Feed | 1 | 2 | 3 | 4 | 5 | 6 | 7 | Die |
|---|---|---|---|---|---|---|---|---|---|
| Temperature | N/A | 65 | 65 | 65 | 65 | 15 | 15 | 15 | 25 |
| Set Point (° C.) | | | | | | | | | |

Table 5 depicts the temperature at successive locations along the barrel of a twin screw extrusion system used to process the composition provided in Table 4.

TABLE 6

| | Screw Element | Element |
|---|---|---|
| Barrel location | Type | Number |
| Feed | *GFA-2-30-90 | 1 |
| | GFA-2-20-60 | 2 |
| | KB4-2-20-30° | 3 |
| | KB4-2-20-60° | 4 |
| | GFA-2-20-30 | 5 |
| | KB4-2-20-90° | 6 |
| | GFA-2-30-90 | 7 |
| | KB 4-2-20-30° | 8 |
| | KB4-2-20-60° | 9 |
| | KB4-2-20-60° | 10 |
| | KB4-2-20-90° | 11 |
| | GFA-2-20-30 | 12 |
| | KB 4-2-20-30° | 13 |
| | KB4-2-20-60° | 14 |
| | KB4-2-20-60° | 15 |
| | GFA-2-20-30 | 16 |
| | KB 4-2-20-30° | 17 |
| | KB4-2-20-90° | 18 |
| | GFA-2-30-90 | 19 |
| | GFA-2-20-60 | 20 |
| Exit | Die | NA |

Table 6 provides the screw element type at successive locations along the barrel of the twin screw extrusion system used to process the composition provided in Table 5 (i.e. beginning at the feed extending to the barrel exit).
FIG. 2 provides an explanation of screw element type terminology.

The temperature set points in barrel locations one through four are set to the melting point of dalcetrapib to ensure that within this region of the barrel the crystalline API is melted, i.e. converted to a liquid state. Within this region, kneading elements (element numbers 3, 4, 6, 8, 10, and 11 are incorporated into the screw design to promote melting of the API and thorough mixing with the molten polymer. Dalcetrapib and poloxamer 188 are completely miscible at 60:25 and 70:10 ratios at 65° C. Miscibility of the API and the polymer ensures molecular mixing which is critical to controlling dalcetrapib crystallization in the subsequent “crystallization region” of the extruder barrel.

The temperature set points are 15° C. at barrel blocks five through seven for the purpose of shock-cooling the molten composite. Rapid cooling in this fashion promotes sudden phase separation of dalcetrapib from the molten polymer. Sudden phase separation promotes the formation of numerous dalcetrapib crystal nuclei which are the seeds for crystal growth. Considering that the reservoir of free dalcetrapib molecules is finite, it is understood that as the number of seeds increase with which free molecules can adhere to during the crystallization process, the size of the crystals formed at the point where the free molecules are exhausted with correspondingly decrease. Therefore, shock cooling in this manner to promote extensive seed formation is essential to achieving fine particles of crystalline dalcetrapib.

The kneading elements incorporated into the screw design at the crystallization region of the extruder barrel (element numbers 14, 15, 17 and 18) act to shear the semi-molten composite via rotation of the screw which provides the mixing function necessary to disperse dalcetrapib crystal seeds throughout the bulk fluid and accelerate crystal formation. By this mixing action of the screw extrusion system the crystallization process is able to be completed on the order of minutes. Conversely, crystallization of dalcetrapib from a stagnant super-cooled melt would require on the order of hours to complete.

### Product Collection

At the exit of the barrel through the die, crystallization of dalcetrapib is near complete and consequently the extrudate is a solid mass which can be easily handled by typical equipment designed to take-off extruded products. In this case, the extrudate is transported from the die exit by a typical belt conveyor to an in-line pelletizer (BT-25 Strand Pelletizer, Bay Plastics Machinery). Depending on the application, the pellets can then be milled using a standard hammer mill and incorporated into a blend for encapsulation, tableting, etc.

### Example 6

### X-Ray Diffraction Analysis of a Crystalline Solid Dispersion of Dalcetrapib in a Poloxamer 188/D-Mannitol Matrix

X-ray diffraction (XRD) analysis was performed on bulk dalcetrapib and the compositions produced according to Example 5 to confirm the crystallinity and polymorph of the API following the HME process.

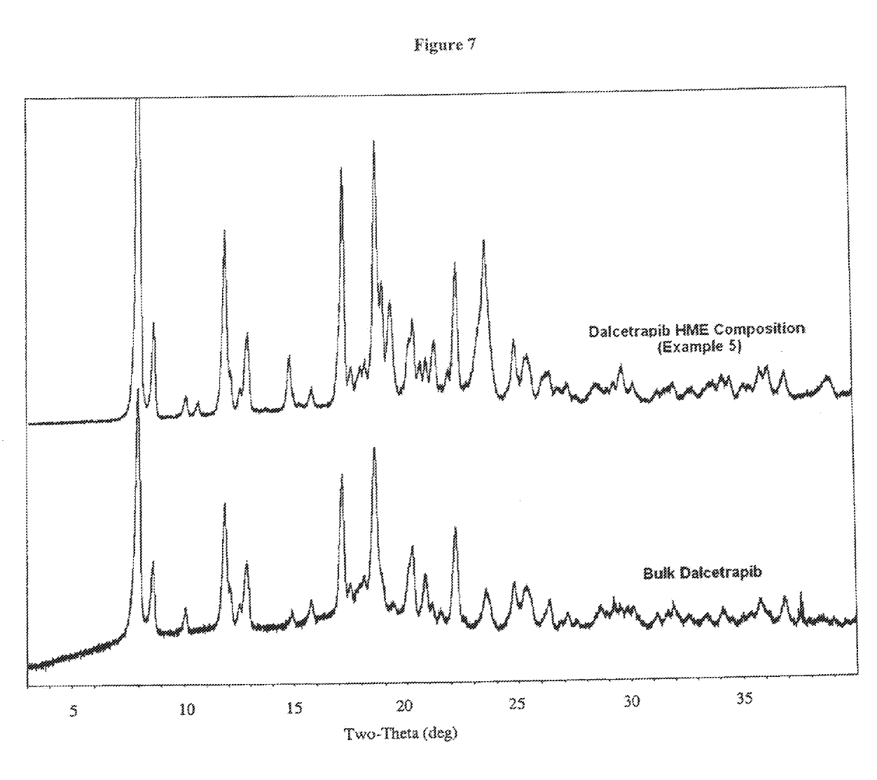

XRD analysis was performed using a Bruker D8 XRD Model D8 Advance x-ray diffractometer. Powder samples were smoothly packed into an aluminum sample holder and loaded onto the sample stage for analysis. The results of this analysis are presented in FIG. 7 which shows an x-ray diffraction pattern representative of the compositions described in Example 5 (both compositions exhibit similar patterns) compared to that of bulk dalcetrapib. It is seen in this figure that the x-ray diffraction pattern of the HME compositions contains the unique peak pattern of the bulk API. Additional peaks seen in the pattern for the HME are attributed to poloxamer 188 and D-mannitol. This XRD analysis confirms that dalcetrapib contained in the HME compositions is substantially crystalline and the crystalline polymorph is identical to that of the bulk API. Thus, it is demonstrated that dalcetrapib is completely recrystallized by the extrusion process following the initial melt transition, and the final crystalline form is identical to that of the bulk API.

### Example 7

### Particle Size Analysis of Crystalline Solid Dispersions of Dalcetrapib in a Poloxamer 188/D-Mannitol Matrix

The particle size distribution of dalcetrapib crystals in the matrices of hot-melt extruded compositions produced according to Example 5 was determined according to the following method:

A Malvern MasterSizer® 2000 was used for particle size measurement. The Fraunhofer® optical model employed for analysis. The sample handling unit was a Hydro 2000S sonicator: Elma Model 9331. Sample measurement time was 20,000 snaps. The sample background time was 20,000 snaps. The dispersant media was 0.1N HCl, and the pump/stir speed was 2000 RPM

Sample preparation was as follows: About 10-15 mg of the sample was weighed in a 20 mL scintillation vial and 10 mL of de-ionized 0.1N HCl was added. The sample was vortexed for 15 seconds and then sonicated for 10 minutes at 100% power.

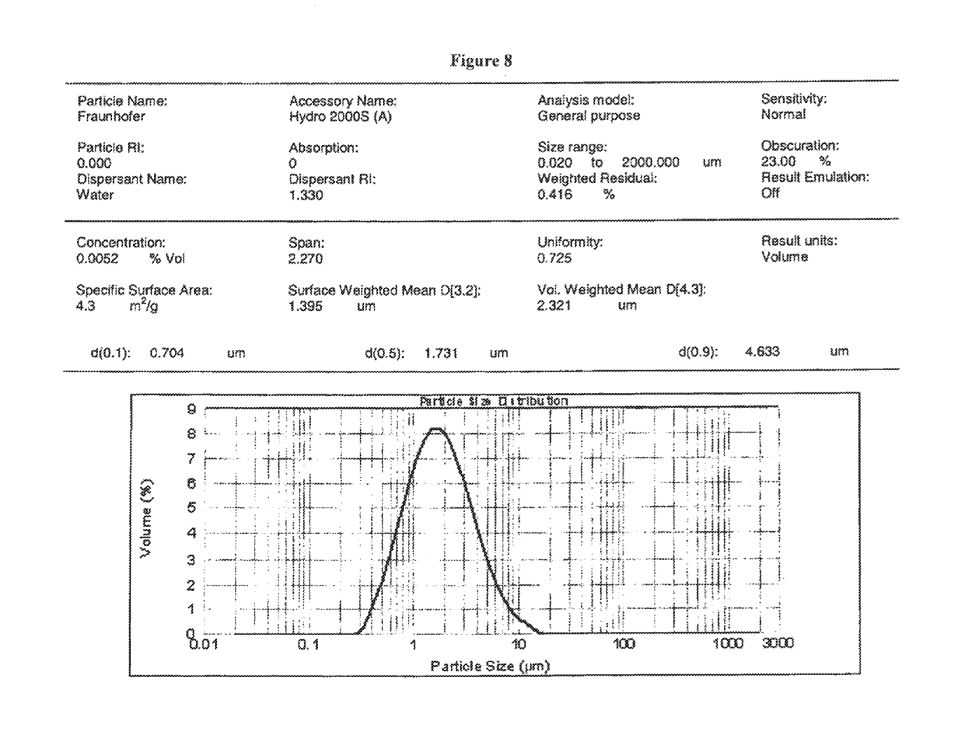

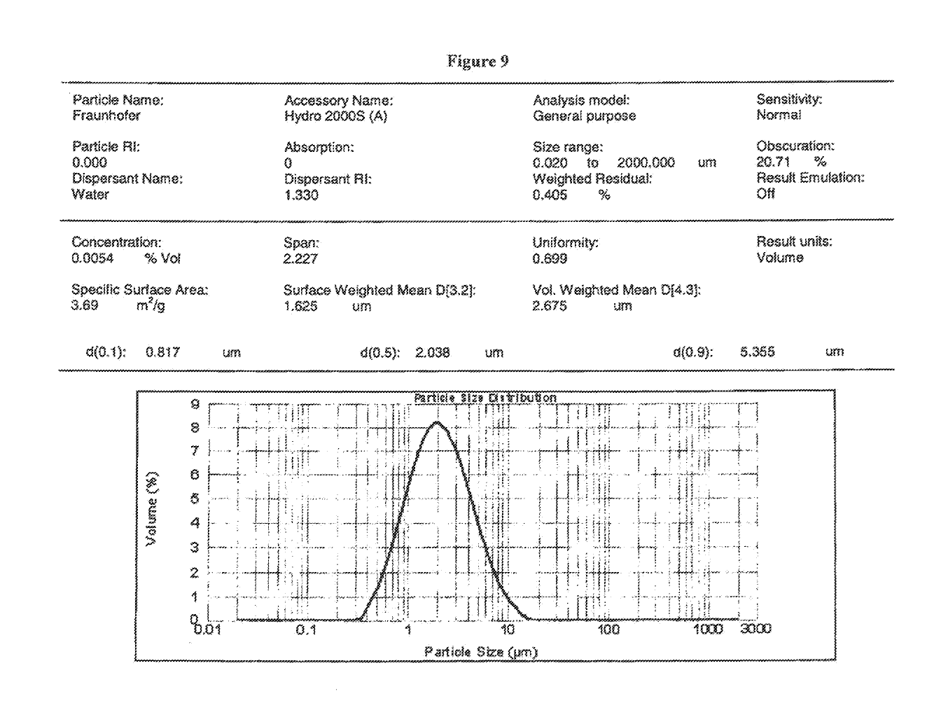

FIG. 8 provides the particle size distribution for the composition containing 60% dalcetrapib described in Example 5. The D(0.1), D(0.5). and D(0.9) values for this composition are 0.704, 1.731, and 4.633 μm respectively. FIG. 9 provides the particle size distribution for the composition containing 70% dalcetrapib described in Example 5. The D(0.1), D(0.5), and D(0.9) values for this composition are 0.817, 2.038, and 5.355 μm, respectively. As is shown in FIG. 4, the mean D(0.1), D(0.5), and D(0.9) values for bulk dalcetrapib are 1.493, 12.317, and 28.828 μm, respectively. Hence, it is demonstrated that significant dalcetrapib particle size reduction is achieved by the HME process described in Example 5 for both compositions.

### Example 8

### Dissolution Analysis of a Crystalline Solid Dispersions with 60% and 70% (w/w) Loading of Dalcetrapib in a Poloxamer 188/D-Mannitol Matrix

Dissolution analysis of the dalcetrapib HME compositions produced according to Example 5 was conducted by the following method

USP Apparatus II (paddle) dissolution testing was conducted using a Distek Evolution 6300 dissolution tester (Distek Inc., North Brunswick, N.J., USA) at a paddle speed of 75 RPM. The dissolution media was 1000 mL of 0.1 N HCl containing 0.75% HTAB (hexadecyltrimethylammonium bromide) equilibrated at 37°±0.5° C. Six replicate samples equivalent to 300 mg dalcetrapib were tested simultaneously. The mean concentration value of these six samples was calculated and reported for each time point. Sample concentrations were determined using an online fiber optic UV detection at 248 nm (Rainbow Dynamic Dissolution Monitor System, Delphian Technology, Woburn, Mass., USA).

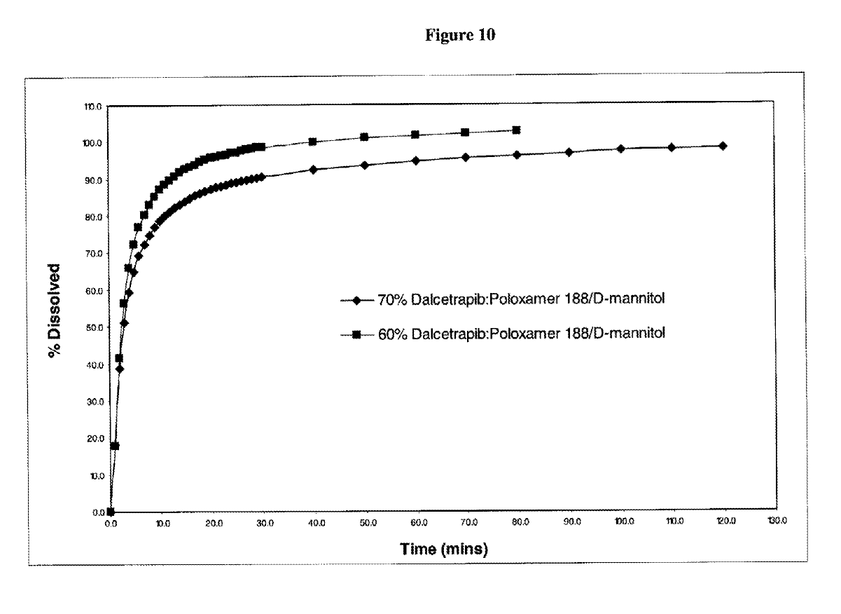

The results of the dissolution analysis of the HME compositions produced according to Example 5 are presented in FIG. 10 which shows that the HME compositions produced according to Example 5 exhibit very rapid dissolution profiles for both the 60% and 70% (w/w) drug load formulations achieving approximately 80% and 90% dissolved in ten minutes, respectively. As described previously in Example 4, the surprisingly rapid dissolution profiles are the results of both API particle size reduction and a very intimate association between the drug and the excipient matrix. The extent of the association between the drug and the matrix is unique to this invention and is brought about by the phase transition of the API particles within the excipient matrix during processing.

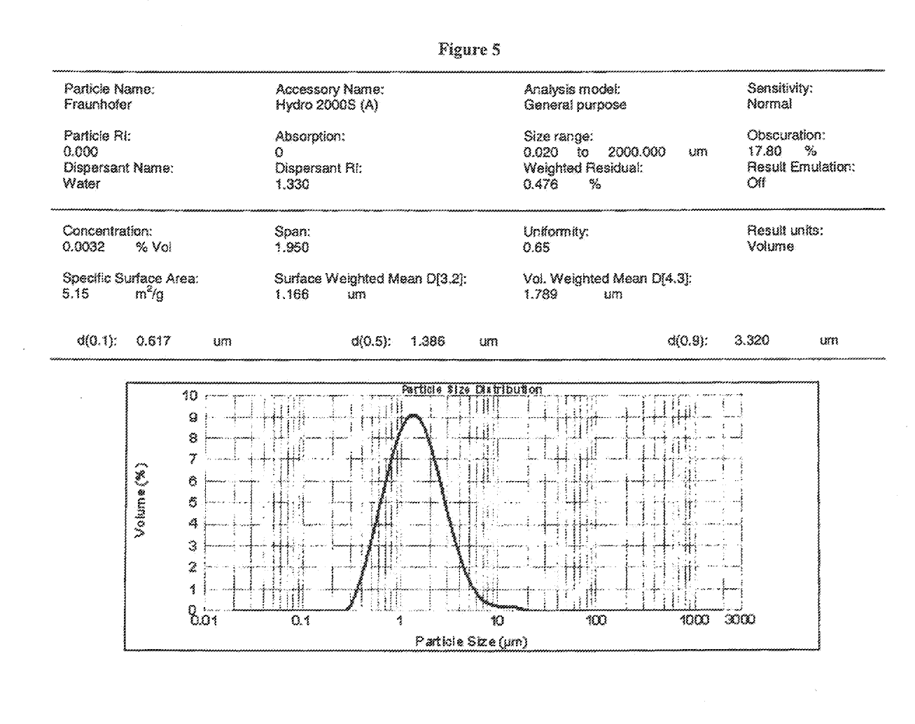

The slightly slower dissolution rate of the 70% drug loading formulation compared to the 60% drug loading formulation is due to greater particle size (see Example 6) as well as the greater hydrophobic content resulting from greater drug loading. The reduced dissolution rate of the current formulation (poloxamer 188/D-mannitol matrix) versus that of Example 1 (amino methacrylate copolymer matrix) can be attributed to greater dalcetrapib particle size in the matrix (FIG. 5 versus FIG. 9), and the slower dissolution rate of poloxamer 188 versus amino methacrylate copolymer. Although the dissolution rate of the compositions described in Example 5 is slower than that of Example 1, these compositions also exhibit surprisingly rapid dissolution of crystalline dalcetrapib and therefore would be expected to provide enhanced bioavailability.

### Example 9

### Production of Crystalline Solid Dispersions of Dalcetrapib of 60% and 70% (w/w) Drug Loadings in Poloxamer 407/Isomalt Matrix

### Process Steps

### Feeding

The API and the excipients comprising the carrier system can be pre-blended and fed to the extrusion system as a single powder mass, or alternatively each component can be fed individually. In this case, the API and excipient components, in the ratios provided in the table below, are first pre-blended in a suitable powder blender (bin or twin-shell).

TABLE 7
Compositions

| Component | % (w/w) | % (w/w) |
|---|---|---|
| Dalcetrapib | 60.0 | 70.0 |
| Poloxamer 407 | 25.0 | 20.0 |
| Isomalt | 15.0 | 10.0 |

Table 7 provides quantitative compositions of a crystalline solid dispersion of dalcetrapib with 60% and 70% (w/w) drug loading in a matrix consisting essentially of poloxamer 407 and isomalt.

### Hot-Melt Extrusion

The resulting powder from blending is then fed into a commonly used twin-screw extrusion system (American Leistritz model Micro-18 lab twin-screw extruder) using a common loss on weight feeder operated at a rate of 20 g/min. The barrel temperature profile and screw configuration are provided below.

TABLE 8

| Barrel location | Feed | 1 | 2 | 3 | 4 | 5 | 6 | 7 | Die |
|---|---|---|---|---|---|---|---|---|---|
| Temperature | N/A | 65 | 65 | 65 | 65 | 15 | 15 | 15 | 25 |
| Set Point (° C.) | | | | | | | | | |

Table 8 depicts the temperature at successive locations along the barrel of a twin screw extrusion system used to process the composition provided in Table 7.

TABLE 9

| | Screw Element | Element |
|---|---|---|
| Barrel location | Type | Number |
| Feed | *GFA-2-30-90 | 1 |
| | GFA-2-20-60 | 2 |
| | KB4-2-20-30° | 3 |
| | KB4-2-20-60° | 4 |
| | GFA-2-20-30 | 5 |
| | KB4-2-20-90° | 6 |
| | GFA-2-30-90 | 7 |
| | KB 4-2-20-30° | 8 |
| | KB4-2-20-60° | 9 |
| | KB4-2-20-60° | 10 |
| | KB4-2-20-90° | 11 |
| | GFA-2-20-30 | 12 |
| | KB 4-2-20-30° | 13 |
| | KB4-2-20-60° | 14 |
| | KB4-2-20-60° | 15 |
| | GFA-2-20-30 | 16 |
| | KB 4-2-20-30° | 17 |
| | KB4-2-20-90° | 18 |
| | GFA-2-30-90 | 19 |
| | GFA-2-20-60 | 20 |
| Exit | Die | NA |

Table 9 provides the screw element type at successive locations along the barrel of the twin screw extrusion system used to process the composition provided in Table 8 (i.e. beginning at the feed extending to the barrel exit).
FIG. 2 provides an explanation of screw element type terminology.

The temperature set points in barrel locations one through four are set to the melting point of dalcetrapib to ensure that within this region of the barrel the crystalline API is melted, i.e. converted to a liquid state. Within this region, kneading elements (element numbers 3, 4, 6, 8, 10, and 11 are incorporated into the screw design to promote melting of the API and thorough mixing with the molten polymer. Dalcetrapib and poloxamer 407 are completely miscible at 60:25 and 70:10 ratios at 65° C. Miscibility of the API and the polymer ensures molecular mixing which is critical to controlling dalcetrapib crystallization in the subsequent “crystallization region” of the extruder barrel.

The temperature set points are 15° C. at barrel blocks five through seven for the purpose of shock-cooling the molten composite. Rapid cooling in this fashion promotes sudden phase separation of dalcetrapib from the molten polymer. Sudden phase separation promotes the formation of numerous dalcetrapib crystal nuclei which are the seeds for crystal growth. Considering that the reservoir of free dalcetrapib molecules is finite, it is understood that as the number of seeds increase with which free molecules can adhere to during the crystallization process, the size of the crystals formed at the point where the free molecules are exhausted with correspondingly decrease. Therefore, shock cooling in this manner to promote extensive seed formation is essential to achieving fine particles of crystalline dalcetrapib.

The kneading elements incorporated into the screw design at the crystallization region of the extruder barrel (element numbers 14, 15, 17 and 18) act to shear the semi-molten composite via rotation of the screw which provides the mixing function necessary to disperse dalcetrapib crystal seeds throughout the bulk fluid and accelerate crystal formation. By this mixing action of the screw extrusion system the crystallization process is able to be completed on the order of minutes. Conversely, crystallization of dalcetrapib from a stagnant super-cooled melt would require on the order of hours to complete.

### Product Collection

At the exit of the barrel through the die, crystallization of dalcetrapib is near complete and consequently the extrudate is a solid mass which can be easily handled by typical equipment designed to take-off extruded products. In this case, the extrudate is transported from the die exit by a typical belt conveyor to an in-line pelletizer (BT-25 Strand Pelletizer, Bay Plastics Machinery). Depending on the application, the pellets can then be milled using a standard hammer mill and incorporated into a blend for encapsulation, tableting, etc.

### Product Properties

The compositions produced according to the procedure described above both exhibited an x-ray diffraction pattern indicating complete recrystallization to the stable polymorph of dalcetrapib was achieved by the process. Particle size reduction of dalcetrapib similar to that of the previous examples was also achieved for these compositions.

### Example 10

### Dissolution Analysis of a Crystalline Solid Dispersions with 60% and 70% Loading of Dalcetrapib in a Poloxamer 407/Isomalt Matrix

Dissolution analysis of the dalcetrapib HME compositions produced according to Example 9 was conducted by the following method

USP Apparatus II (paddle) dissolution testing was conducted using a Distek Evolution 6300 dissolution tester (Distek Inc., North Brunswick, N.J., USA) at a paddle speed of 75 RPM. The dissolution media was 1000 mL of 0.1 N HCl containing 0.75% HTAB equilibrated at 37°±0.5° C. Six replicate samples equivalent to 300 mg dalcetrapib were tested simultaneously. The mean concentration value of these six samples was calculated and reported for each time point. Sample concentrations were determined using an online fiber optic UV detection at 248 nm (Rainbow Dynamic Dissolution Monitor System, Delphian Technology, Woburn, Mass., USA).

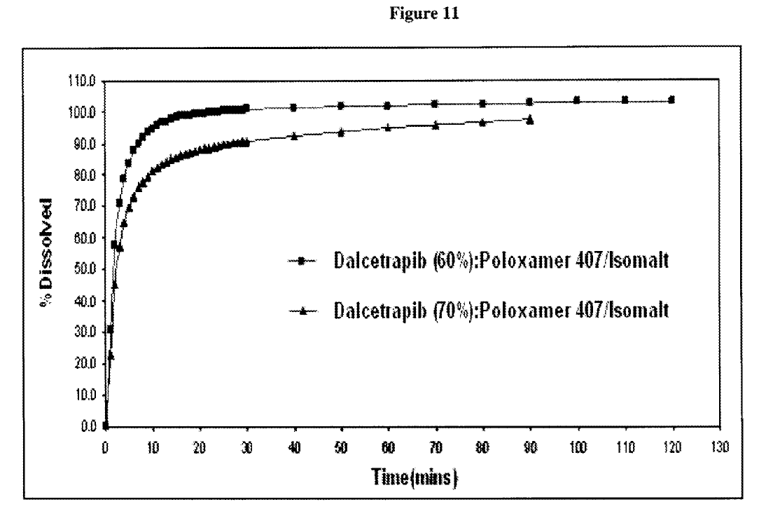

The results of the dissolution analysis of the HME compositions produced according to Example 9 are presented in FIG. 11 which shows that the HME compositions produced according to Example 9 exhibit very rapid dissolution profiles for both the 60% and 70% drug load formulations achieving approximately 80% and 90% dissolved in ten minutes, respectively. As described previously in Example 4, the surprisingly rapid dissolution profiles are the result of both API particle size reduction and a very intimate association between the drug and the excipient matrix. The extent of the association between the drug and the matrix is unique to this invention and is brought about by the phase transition of the API particles within the excipient matrix during processing.

The slightly slower dissolution rate of the 70% drug loading formulation compared to the 60% drug loading formulation is likely due to greater particle size as well as the greater hydrophobic content resulting from greater drug loading. The reduced dissolution rate of the current formulation (poloxamer 407/Isomalt) versus that of Example 1 (amino methacrylate copolymer matrix) can be attributed to greater dalcetrapib particle size in the matrix and the slower dissolution rate of poloxamer 407 versus amino methacrylate copolymer. Although the dissolution rate of the compositions described in Example 9 are less rapid than that of Example 1, these compositions also exhibit surprisingly rapid dissolution of crystalline dalcetrapib and therefore would be expected to provide enhanced bioavailability.

1. A hot-melt extruded pharmaceutical composition comprising a hydrophobic compound with a melting point less than 250° C. and a glass transition temperature below 45° C. made by a process comprising: (1) feeding the compound contemporaneously with excipients to the extruder barrel of an extruder, (2) melting the compound by the application of heat and shear by the shearing action of extruder screws rendering the compound non-crystalline, (3) mixing the melted compound and excipients in the first one-fourth to one-half of the extruder barrel by the shearing action of extruder screws, (4) subsequently recrystallizing the compound from the molten mixture in the second one-half to three-fourths of the extruder barrel by decreasing the heat and shearing action, and (5) forcing the composition through a shaping die at the end of the extruder; wherein the mean particle diameter of the compound is significantly reduced compared to the original compound before extrusion.

2. The composition of claim 1 wherein the compound in the hot-melt extruded composition has a mean particle diameter of less than 10 microns.

3. The composition of claim 1 wherein the compound in the hot-melt extruded composition has a mean particle diameter of less than 5 microns.

4. The composition of claim 1 wherein the pharmaceutically acceptable carrier comprises at least one thermal binder.

5. The composition of claim 4 wherein the thermal binder is an amoniomethacrylate copolymer.

6. The composition of claim 4 wherein the thermal binder is a polyethylene oxide-polypropylene oxide copolymer.

7. The composition of claim 1 wherein at least one excipient is an antioxidant or wetting agent.

8. The composition of claim 1 wherein at least one excipient is a surfactant or disintegrant.

9. The composition of claim 1 wherein at least one excipient is a filler or stabilizing agent.

10. The composition of claim 1 wherein the compound is a CETP inhibitor.

11. The composition of claim 1 wherein the compound is selected from the group consisting of ibuprofen, ketoprofen, indomethacin, and acetaminophen.

12. The composition of claim 1 wherein the compound is dalcetrapib.

13. The composition of claim 1 comprising dalcetrapib, amino methacrylate copolymer and fumed silica.

14. The composition of claim 1 comprising about 70% dalcetrapib, about 29.75% amino methacrylate copolymer, and about 0.25% fumed silica.

15. The composition of claim 1 comprising dalcetrapib, poloxamer 407, and D-mannitol.

16. The composition of claim 1 comprising about 60% dalcetrapib, about 25% poloxamer 188, and about 15% D-mannitol.

17. The composition of claim 1 comprising about 70% dalcetrapib, about 20% poloxamer 188, and about 10% D-mannitol.

18. The composition of claim 1 comprising dalcetrapib, poloxamer 407, and isomalt.

19. The composition of claim 1 comprising about 60% dalcetrapib, about 25% poloxamer 407, and about 15% isomalt.

20. The composition of claim 1 comprising about 70% dalcetrapib, about 20% poloxamer 407, and about 10% isomalt.
